SEQUENCE LISTING (12 sequences)

SEQ ID NO: 1 - DNA, 21 bp, Artificial Sequence
  primer
tgccatttttaatgtctttag

SEQ ID NO: 2 - DNA, 19 bp, Artificial Sequence
  primer
tggaattccggctacctac

SEQ ID NO: 3 - DNA, 21 bp, Artificial Sequence
  probe
agacgctaccatggctatcgc

SEQ ID NO: 4 - DNA, 25 bp, Artificial Sequence
  primer
tcccaagaagctgttcagaatcaga

SEQ ID NO: 5 - DNA, 22 bp, Artificial Sequence
  primer
cactccgtggacagatttgtca

SEQ ID NO: 6 - DNA, 21 bp, Artificial Sequence
  probe
tgagccgcaacttcgggatga

SEQ ID NO: 7 - DNA, 21 bp, Artificial Sequence
  primer
cgggactttgcaagtggtgaa

SEQ ID NO: 8 - DNA, 24 bp, Artificial Sequence
  primer
acgcacagttcatagagataacct

SEQ ID NO: 9 - DNA, 20 bp, Artificial Sequence
  probe
cccacctctggcaaccgggt

SEQ ID NO: 10 - DNA, 20 bp, Artificial Sequence
  primer
ggaccatcgagcagatcacc

SEQ ID NO: 11 - DNA, 18 bp, Artificial Sequence
  primer
cccatcggcaccatcgca

SEQ ID NO: 12 - DNA, 27 bp, Artificial Sequence
  probe
tgccgatatgctggcgatgtccgccaa

(19) United States
(12) Patent Application Publication
(10) Pub. No.: US 2016/0348153 A1
(43) Pub. Date: Dec. 1, 2016

(54) EXTRACTION AND PRESERVATION OF NUCLEIC ACID MOLECULES FROM PATHOGENS

(71) Applicant: THE UNITED STATES OF AMERICA, as represented by the Secretary, Department of Health and Human Serv, Bethesda, MD (US)

(72) Inventors: Jothikumar Narayanan, Atlanta, GA (US); Vincent Hill, Decatur, GA (US); Jan Vinje, Atlanta, GA (US); Theresa Cromeans, Atlanta, GA (US)

(73) Assignee: THE UNITED STATES OF AMERICA, as represented by the Secretary, Department of Health and Human Serv, Bethesda, MD (US)

(21) Appl. No.: 15/169,404

(22) Filed: May 31, 2016

Related U.S. Application Data
(60) Provisional application No. 62/168,582, filed on May 29, 2015.

(51) Int. Cl. C12Q 1/68; C12N 15/10; C12Q 1/70

(52) U.S. Cl. CPC C12Q 1/6806; C12Q 1/701; C12Q 1/689; C12N 15/1006

(57) ABSTRACT

This application provides a novel lysis buffer that can be used for storage of nucleic acid molecules on a solid support, and methods of storing nucleic acid molecules on a solid support and extracting nucleic acid molecules from a solid support.

### CROSS-REFERENCE TO RELATED APPLICATION

This application claims priority to U.S. Provisional Application No. 62/168,582 filed May 29, 2015, herein incorporated by reference.

### FIELD

This application provides a novel lysis buffer and methods of its use for storing and extracting nucleic acid molecules from pathogens, for example on a solid support, which also allows for safe transport of the nucleic acid molecules (for example at ambient temperatures).

### BACKGROUND

Molecular testing is a rapid approach for investigating global public health and environmental crises that generate biological specimens for diagnosis. When molecular laboratory testing facilities are not readily available, biological specimens must be stored and/or transported to other laboratories, often out of country. Shipment of frozen samples is difficult, expensive and stringent shipping regulations apply. This often delays or prevents testing of samples that are critical for understanding and responding to public health crises.

There is an increasing interest in developing and evaluating storage and transport media that can preserve nucleic acid molecules for molecular testing. Samples may be stored either in a liquid or dry state. However, transport of pathogens is a liquid state is problematic as it may be difficult to inactivate the pathogens in the liquid sample. Several commercial filter paper cards composed of pure cellulose paper or chemically treated paper (e.g., FTA® card) are available for transport of samples. The exact treatment formulas for these products is often proprietary, but the general mechanism of action is that applied cells are lysed, pathogens inactivated, and nucleic acid stabilized for extended storage times. However, such cards lack internal controls, and are only able to detect one type of pathogen (e.g., primarily DNA viruses).

### SUMMARY

The present disclosure provides buffers, which in some examples can be used to aid in the storage and extraction of nucleic acid molecules on a solid support.

In one example, the buffer comprises or consists of:

1 M to 5 M guanidine thiocyanate (GuSCN) in Tris EDTA (TE) Buffer (wherein the TE buffer can comprise or consist of 1 to 50 mM Tris (such as 5 mM to 20 mM, 5 mM to 30 mM, 5 mM to 15 mM, or 1 mM to 20 mM Tris), 0.01 to 5 mM EDTA (such as 0.5 mM to 5 mM, 0.5 mM to 3 mM, 0.5 mM to 1 mM, or 1 mM to 3 mM EDTA), pH 6.0 to 9.5, such as pH 7.5 to 8.5, pH 7 to pH 8.5, pH 7 to 8, or pH 7 to 9);

0.5% to 4% polyethylene glycol 8000 (such as 1% to 4%, 2% to 4%, 3% to 4%);

0.1 M to 2 M NaCl (such as 0.2 M to 0.5 M, 0.2 M to 0.4M, 0.2 M to 1 M, or 0.5 M to 1 M);

0.05 M to 1 M NaOAC (such as 0.05 M to 0.2 M, 0.05 M to 0.5M, 0.1 M to 0.2 M, or 0.1 M to 0.5 M);

0.1% to 1% of dithioerythritol (DTE) (such as 0.1% to 0.5%, 0.1% to 0.4%, 0.2% to 0.6%, or 0.15% to 0.25%);

0.1% to 2% Na₂SO₃ (such as 0.1% to 0.5%, 0.1% to 1%, 0.2% to 0.6%, or 0.2% to 1%);

1 μg/ml to 100 μg/ml polyadenylic acid 5′ (PolyA) (such as 1 μg/ml to 20 μg/ml, 10 μg/ml to 20 μg/ml %, 10 μg/ml to 50 μg/ml, or 15 μg/ml to 30 μg/ml);

0.01% to 0.5% sodium dodecyl sulfate (SDS) (such as 0.01% to 0.05%, 0.01% to 0.1%, 0.02% to 0.06%, or 0.02% to 0.04%);

0.1% to 2% Tween® 20 detergent (polysorbate 20) (such as 0.1% to 0.5%, 0.1% to 1%, 0.2% to 0.6%, or 0.2% to 1%); and

water, such as nuclease free water.

In one example, the buffer, when at a volume of 250 ml, comprises or consists of:

132 grams of guanidine thiocyanate (GuSCN);

50 mL of Tris EDTA (TE) Buffer, pH 8;

50 mL of 20% polyethylene glycol 8000;

12 mL of 5M NaCl;

12 mL of 3M NaOAC, pH 5;

0.5 g of dithioerythritol (DTE);

1 g of Na₂SO₃;

2.2 ml of polyadenylic acid 5′ (PolyA, at 2 mg/mL);

250 μl of 20% sodium dodecyl sulfate (SDS);

1 mL of Tween® 20 detergent (polysorbate 20); and

remaining volume of water, such as nuclease free water.

In one example, the buffer comprises or consists of:

4.5 M guanidine thiocyanate (GuSCN) in Tris EDTA (TE) Buffer, pH 8 (wherein Tris is 10 mM and EDTA is 1 mM);

4% polyethylene glycol 8000;

0.24 M NaCl;

0.14 M NaOAC;

0.2% of dithioerythritol (DTE);

0.4% Na₂SO₃;

17.6 μg/ml polyadenylic acid 5′ (PolyA);

0.02% sodium dodecyl sulfate (SDS);

0.4% Tween® 20 detergent (polysorbate 20); and

water, such as nuclease free water.

One skilled in the art will recognize that the amount of each reagent listed may vary slightly, such as vary by no more than 5%, no more than 4%, no more than 3%, no more than 2%, no more than 1%, no more than 0.5%, or no more than 0.1%.

Also provided are solid supports that include the lysis buffer. For example, the lysis buffer can be applied to a solid support and allowed to dry. In some examples, the solid support further includes one or more controls, such as a control to determine if nucleic acid molecules were properly extracted, to determine if subsequent analysis (e.g., PCR) of the nucleic acids was properly performed, or combinations thereof. Thus, in some examples, the solid support includes lysis buffer (e.g., dried on the support), and a nucleic acid control (e.g., an RNA bacteriophage MS2 nucleic acid or a DNA bacteriophage PhiX 174). Examples of solid supports include those made from paper, cellulose, nitrocellulose, metal, cardboard, and plastic. In one example, the solid support is a card or disc made of nitrocellulose.

Also provided are methods of analyzing nucleic acid molecules. Exemplary nucleic acid molecules include DNA, RNA, or both. Nucleic acid molecules from any organism can be present on the solid support can be analyzed, such as those from a pathogen (e.g., bacteria, virus, or parasite), mammal (e.g., human or veterinary subject), and the like. Such methods can include contacting a solid support with a test sample, which may contain one or more target nucleic acid molecules, wherein the solid support was previously incubated with the disclosed lysis buffer and allowed to dry, under conditions that allow stabilization of the nucleic acids subsequently applied to the solid support. If desired, the sample can be stored on the solid support, for example for a period of days, weeks or months, for example at ambient temperatures. The methods further include extracting the nucleic acid molecules from the solid support, for example by heating the solid support (e.g., in an aqueous solution such as water), for example at a temperature of at least 80° C., at least 90° C., or at least 95° C., such as 90 to 100° C., such as at 95° C. The extracted nucleic acids can then be analyzed, for example by PCR, sequencing, both or other methods.

The foregoing and other objects and features of the disclosure will become more apparent from the following detailed description, which proceeds with reference to the accompanying figures.

### BRIEF DESCRIPTION OF THE DRAWINGS

FIGS. 1A-1F are graphs showing the stability of viral nucleic acids (A, rotavirus, B, hepatitis A virus, C, Adenovirus, D, Salmonella, E, Crytosporidum parvum, and F, MS2 RNA) stored in water (W) or lysis buffer described in Example 1 (L) at 4° C. or 35° C. Samples were stored at for up to 416 days. In FIG. 1F, starting Ct values for lysis buffer and water-only samples differed because of RNA degradation that occurred during the time when mastermix was being prepared and template added; the same RNA amount was added to all storage tubes. “Missing” Ct values reflect negative reactions.

FIG. 2 is a schematic drawing showing processing of a solid support (16 mm paper disk) for storing and extracting nucleic acids applied thereto.

FIG. 3 is a schematic drawing showing application of a sample to the solid support, and storage of the nucleic acids in the sample on the solid support (here, a paper disc).

### SEQUENCE LISTING

The nucleotide sequences of the nucleic acids described herein are shown using standard letter abbreviations for nucleotide bases. Only one strand of each nucleic acid sequence is shown, but the complementary strand is understood as included by any reference to the displayed strand. The sequence listing generated on May 31, 2016 (2.37 kb) and submitted herewith is herein incorporated by reference.

SEQ ID NO: 1 is the nucleic acid sequence for an MS2 forward primer.

SEQ ID NO: 2 is the nucleic acid sequence for an MS2 reverse primer.

SEQ ID NO: 3 is the nucleic acid sequence for an MS2 probe.

SEQ ID NO: 4 is the nucleic acid sequence for a PhiX174 forward primer.

SEQ ID NO: 5 is the nucleic acid sequence for a PhiX174 reverse primer.

SEQ ID NO: 6 is the nucleic acid sequence for a PhiX174 probe.

SEQ ID NO: 7 is the nucleic acid sequence for an E. coli uidA (T6) gene forward primer.

SEQ ID NO: 8 is the nucleic acid sequence for an E. coli uidA (T6) gene reverse primer.

SEQ ID NO: 9 is the nucleic acid sequence for an E. coli uidA (T6) gene probe. SEQ ID NO: 10 is the nucleic acid sequence for an E. coli tnaA (T10) gene forward primer.

SEQ ID NO: 11 is the nucleic acid sequence for an E. coli tnaA (T10) gene reverse primer.

SEQ ID NO: 12 is the nucleic acid sequence for an E. coli tnaA (T10) gene probe.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. All references, patents, and patent applications referred to herein are incorporated by reference.

Contact: Placement in direct physical association, including a solid or a liquid form. Contacting can occur, for example, by adding a reagent (such as a sample or lysis buffer) to a solid support.

Nucleic acid molecule: A deoxyribonucleotide or ribonucleotide polymer, which can include analogues of natural nucleotides that hybridize to nucleic acid molecules in a manner similar to naturally occurring nucleotides. In a particular example, a nucleic acid molecule is a single-stranded (ss) DNA or RNA molecule, such as a cDNA, mRNA, or transcription product. In another particular example, a nucleic acid molecule is a double-stranded (ds) molecule, such as cellular genomic DNA or viral genomic RNA.

Pathogens/Microbes: Infectious agents, which include, but are not limited to, viruses, bacteria, fungi, nematodes, and protozoa. A non-limiting list of pathogens whose nucleic acid molecules can be analyzed using the disclosed methods, and thus can be applied to a solid support (e.g., may be present in a sample applied to the solid support), are provided below.

Viruses include positive-strand RNA viruses and negative-strand RNA viruses. Exemplary positive-strand RNA viruses include, but are not limited to: Picornaviruses (such as Aphthoviridae [for example foot-and-mouth-disease virus (FMDV)] and Hepatovirus [such as Hepatitis A, B, or C virus]), Cardioviridae; Enteroviridae (such as Coxsackie viruses, Echoviruses, Enteroviruses, and Polioviruses); Rhinoviridae (Rhinoviruses)); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, Zika virus, yellow fever virus, and Japanese encephalitis virus); Caliciviridae (which includes norovirus [such as human and murine norovirus] and sapovirus); and Coronaviruses (examples of which include SARS coronaviruses, such as the Urbani strain, and MERS coronaviruses). Exemplary negative-strand RNA viruses include, but are not limited to: Orthomyxyoviruses (such as the influenza virus), Rhabdoviruses (such as Rabies virus), Ebola virus, and Paramyxoviruses (examples of which include measles virus, respiratory syncytial virus, and parainfluenza viruses).

Viruses also include DNA viruses. DNA viruses include, but are not limited to: Herpesviruses (such as Varicella-zoster virus, for example the Oka strain; cytomegalovirus; and Herpes simplex virus (HSV) types 1 and 2), Adenoviruses (such as adenovirus type 1, adenovirus type 40, and adenovirus type 41), Poxviruses (such as Vaccinia virus), and Parvoviruses (such as Parvovirus B 19).

Another group of viruses includes retroviruses. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

Thus, one example, nucleic acid molecules from a virus are analyzed, such as one or more of the following: HIV; hepatitis A virus (HAV); Hepatitis B (HB) virus; Hepatitis C (HC) virus; Hepatitis D (HD) virus; Hepatitis E virus; a respiratory virus (such as influenza A & B, respiratory syncytial virus, human parainfluenza virus, or human metapneumovirus), Zika virus, Ebola virus, measles virus, or West Nile Virus.

Pathogens also include bacteria. Bacteria can be classified as gram-negative or gram-positive. Exemplary gram-negative bacteria include, but are not limited to: Escherichia coli (e.g., K-12 and O157:H7), Shigella dysenteriae, and Vibrio cholerae. Exemplary gram-positive bacteria include, but are not limited to: Bacillus anthracis, Staphylococcus aureus, Listeria, pneumococcus, gonococcus, and streptococcal meningitis. In one example, the bacteria include one or more of the following: Group A Streptococcus; Group B Streptococcus; Helicobacter pylori; Methicillin-resistant Staphylococcus aureus; Vancomycin-resistant enterococci; Clostridium difficile; Clostridium perfringens; E. coli (e.g., Shiga toxin producing strains); Listeria; Salmonella (e.g., S. enterica subsp. enterica); Campylobacter; B. anthracis (such as spores); Chlamydia trachomatis; and Neisseria gonorrhoeae.

Protozoa, nemotodes, and fungi are also types of pathogens that can be analyzed using the disclosed solid supports. Exemplary protozoa include, but are not limited to, Plasmodium (e.g., Plasmodium falciparum to diagnose malaria), Leishmania, Acanthamoeba, Giardia (e.g., Giardia intestinalis, Giardia duodenalis), Entamoeba, Cryptosporidium (e.g., Cryptosporidium parvum), Isospora, Balantidium, Trichomonas, Trypanosoma (e.g., Trypanosoma brucei), Naegleria, schistosomes, Toxoplasma and free living amoebas. Exemplary fungi include, but are not limited to, Coccidiodes immitis and Blastomyces dermatitidis.

In one example, nucleic acid molecules from one or more bacterial spores are analyzed. For example, the genus of Bacillus and Clostridium bacteria produce spores. Thus, nucleic acid molecules from C. botulinum, C. perfringens, B. cereus, and B. anthracis spores (e.g., anthrax spores) can be analyzed using the disclosed solid supports. One will also recognize that nucleic acid molecules from spores from green plants can also be analyzed using the disclosed solid supports.

In one example, nucleic acid molecules from protozoan cysts are analyzed using the disclosed solid supports. For example, the genus of Cryptosporidium and Giardia produce cysts or oocysts. Thus, nucleic acid molecules from C. parvum oocysts and Giardia duodenalis cysts can be analyzed using the disclosed solid supports.

In one example, nucleic acid molecules from a stool sample are analyzed using the disclosed solid supports, such as enteropathogens (e.g., norovirus, rotavirus, enterovirus, and/or parasites). In one example, nucleic acid molecules from a respiratory swab sample are analyzed using the disclosed solid supports, such as influenza, rhinovirus, RSV, and/or adenovirus.

Sample: Biological specimens such as samples containing biomolecules, for example nucleic acid molecules (e.g., genomic DNA, cDNA, RNA, and/or mRNA). Exemplary samples are those containing cells or cell lysates from a subject (and which may contain one or more pathogens), such as peripheral blood (or a fraction thereof such as plasma or serum), urine, saliva, sputum, tissue biopsy, cheek swabs, fecal specimen (e.g., stool sample), respiratory specimen, surgical specimen, fine needle aspirates, amniocentesis samples and autopsy material. Also includes other types of samples, such as environmental samples (e.g., soil, air, water), and food samples. Samples can be applied to a solid support, for example to store nucleic acid molecules present in the sample.

Solid Support: A material to which a nucleic acid molecule can be attached, and in some examples is formed from a water immiscible material. In some examples, suitable characteristics of the material that can be used to form the solid support surface include: being amenable to application and drying of the disclosed lysis buffer, being chemically inert, or both.

The surface of a solid support may be activated by chemical processes that cause covalent linkage of an agent (e.g., nucleic acid molecule) to the support, such as application of the disclosed lysis buffer. However, any other suitable method may be used for immobilizing an agent (e.g., a nucleic acid molecule) to a solid support including, without limitation, ionic interactions, hydrophobic interactions, covalent interactions and the like.

A wide variety of solid supports can be employed in accordance with the present disclosure. Except as otherwise physically constrained, a solid support may be used in any suitable shape, such as films, sheets, strips, discs, or plates, or it may be coated onto or bonded or laminated to appropriate inert carriers, such as paper, glass, plastic films, or fabrics.

In one example the solid support is a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example the membrane comprises nitrocellulose. In a specific example the solid support is an FTA® card.

In some embodiments, porous solid supports, such as nitrocellulose, are in the form of sheets or strips, discs, or cards. The thickness of such sheets, discs, or strips or cards may vary within wide limits, for example, at least 0.01 mm, at least 0.1 mm, or at least 1 mm, for example from about 0.01 to 5 mm, about 0.01 to 2 mm, about 0.01 to 1 mm, about 0.01 to 0.5 mm, about 0.02 to 0.45 mm, from about 0.05 to 0.3 mm, from about 0.075 to 0.25 mm, from about 0.1 to 0.2 mm, or from about 0.11 to 0.15 mm. The pore size of such may similarly vary within wide limits, for example from about 0.025 to 15 microns, or from about 0.1 to 3 microns; however, pore size is not intended to be a limiting factor in selection of the solid support.

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof).

In some examples, the solid support is a microtiter plate, ELISA plate, test tube, inorganic sheets, dipstick, lateral flow device, and the like. In another example the solid support is a nitrocellulose membrane. In another example the format is filter paper. In yet another example the format is a glass slide. In one example, the solid support includes polypropylene thread. One or more polypropylene threads can be affixed to a plastic dipstick-type device; polypropylene membranes can be affixed to glass slides.

Subject: A vertebrate, such as a mammal, for example a human. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, horse, or cow. In some examples, the subject has or is suspected of being infected with a pathogen. Thus, subjects can serve as a source of samples analyzed using the disclosed methods.

Under conditions sufficient for: A phrase that is used to describe any environment that permits the desired activity. An example includes incubating forward and reverse primers with a sample under conditions sufficient to allow amplification of a target nucleic acid molecule in the sample. Another particular example includes conditions sufficient for allowing a lysis buffer and nucleic acid molecules to adhere to a solid support.

### Overview

A new universal lysis buffer is disclosed, which allows the stability and integrity of microbial RNA and DNA to be maintained at different storage temperatures and time in a solid format. It is shown herein that filter paper discs (cards) saturated with the buffer can effectively inactivate pathogens and store nucleic acid molecules (DNA and RNA) from the pathogens at various temperatures, and allow effective removal of the nucleic acid molecules from the card. The removed nucleic acid molecules from the pathogens can then be analyzed, for example using PCR and/or sequencing.

A comparison of the currently available FTA Card (from Whatman/GE) and the disclosed solid support, is shown in Table 1.

TABLE 1
Comparison of Old and New
Solid Supports for Nucleic Acid Storage

| FEATURE | FTA | New Card |
|---|---|---|
| Cost | About $5 | Less than $1 |
| Sensitivity | Less | Greater* |
| Internal Standards | No | Yes |
| Nucleic acid | Mostly for DNA, | DNA AND RNA |
| detection | few RNA reports, | Quantitation evaluated |
| | No quantitation** | for RNA and DNA |
| | | viruses |
| Dessicant Needed | Yes | No |
| Sequence Nucleic | Yes | Yes |
| Acid | | |

*Larger portion card tested, therefore more sensitive (16 mm vs. 3 mm)
**Li et al. (J. Virol. Meth. 186 (2012) 62-67). An optimized method for elution of enteroviral RNA from a cellulose-based substrate. Recovery rate for viral RNA eluted from FTA elute cards was only 6.1%; FTA elute better than FTA classic for RNA according to the company.

### Lysis Buffer

The present disclosure provides a lysis buffer that can be used to store nucleic acid molecules, for example from one or more pathogens. The lysis buffer can be used to store nucleic acid molecules in solution (for example by adding a sample containing pathogen(s) to the buffer), or on a solid support (for example by adding the buffer to the solid support, drying the buffer, and then adding a sample that includes one or more pathogens). In some examples, the lysis buffer also inactivates the pathogens (e.g., virus, such as Ebola) applied to the solid support.

In one example, the buffer comprises or consists of:

1 M to 5 M guanidine thiocyanate (GuSCN) in Tris EDTA (TE) Buffer (wherein the TE buffer can comprise or consist of 1 to 50 mM Tris (such as 5 mM to 20 mM, 5 mM to 30 mM, 5 mM to 15 mM, or 1 mM to 20, for example 1 mM, 5 mM, 10 mM or 20 mMTris), 0.01 to 5 mM EDTA (such as 0.5 mM to 5 mM, 0.5 mM to 3 mM, 0.5 mM to 1 mM, or 1 mM to 3 mM, for example 0.01, 0.05, 0.1, 0.2 or 0.5 mM EDTA), pH 6.0 to 9.5, such as pH 7.5 to 8.5, pH 7 to pH 8.5, pH 7 to 8, or pH 7 to 9, for example pH 6, 6.1, 6.2, 6.3, 6.4, 6.5, 6.6, 6.7, 6.8, 6.9. 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9. 9, 9.1, 9.2, 9.3, 9.4, or 9.5);

0.5% to 4% polyethylene glycol 8000 (such as 1% to 4%, 2% to 4%, 3% to 4%, for example, 0.5%, 1%, 2%, 3% or 4% PEG 8000);

0.1 M to 2 M NaCl (such as 0.2 M to 0.5 M, 0.2 M to 0.4M, 0.2 M to 1 M, or 0.5 M to 1 M, for example 0.1 M, 0.15 M, 0.2 M, 0.24M, 0.3M, 0.5M, 0.8 M, 1 M, or 2M NaCl);

0.05 M to 1 M NaOAC (such as 0.05 M to 0.2 M, 0.05 M to 0.5M, 0.1 M to 0.2 M, or 0.1 M to 0.5 M, for example 0.05 M, 0.1 M, 0.14 M, 0.16M, 0.2M, 0.3M, 0.5M, 0.8 M, 0.9 M, or 1M NaOAC);

0.1% to 1% of dithioerythritol (DTE) (such as 0.1% to 0.5%, 0.1% to 0.4%, 0.2% to 0.6%, or 0.15% to 0.25%, for example 0.1%, 0.15%, 0.2%, 0.3%, 0.4%, 0.5%, or 1% DTE);

0.1% to 2% Na₂SO₃ (such as 0.1% to 0.5%, 0.1% to 1%, 0.2% to 0.6%, or 0.2% to 1%, for example 0.1%, 0.2%, 0.3%, 0.4%, 0.5%, or 1% Na₂SO₃);

1 μg/ml to 100 μg/ml polyadenylic acid 5′ (PolyA) (such as 1 μg/ml to 20 μg/ml, 10 μg/ml to 20 μg/ml %, 10 μg/ml to 50 μg/ml, or 15 μg/ml to 30 μg/ml, for example 10 μg/ml, 15 μg/ml, 17.6 μg/ml, 20 μg/ml, 25 μg/ml, 30 μg/ml, 50 μg/ml, or 75 μg/ml PolyA);

0.01% to 0.5% sodium dodecyl sulfate (SDS) (such as 0.01% to 0.05%, 0.01% to 0.1%, 0.02% to 0.06%, or 0.02% to 0.04%, for example 0.01%, 0.015%, 0.02%, 0.03%, 0.04%, 0.05%, 0.1%, or 0.5% SDS);

0.1% to 2% Tween® 20 detergent (polysorbate 20) (such as 0.1% to 0.5%, 0.1% to 1%, 0.2% to 0.6%, or 0.2% to 1%, for example 0.1%, 0.15%, 0.2%, 0.3%, 0.4%, 0.5%, 1%, or 2% polysorbate 20); and water, such as nuclease free water.

In some examples, the lysis buffer, when at a volume of 250 ml, includes or consists of:

132 grams of guanidine thiocyanate (GuSCN);

50 mL of Tris EDTA (TE)Buffer, pH 8;

50 mL of 20% polyethylene glycol 8000;

12 mL of 5M NaCl;

12 mL of 3M NaOAC, pH 5;

0.5 g of dithioerythritol (DTE);

1 g of Na₂SO₃;

2.2 ml of polyadenylic acid 5′ (PolyA, at 2 mg/mL);

250 μl of 20% sodium dodecyl sulfate (SDS);

1 mL of Tween® 20 detergent (polysorbate 20); and

remaining volume of nuclease free water.

In some examples, the lysis buffer includes or consists of:

4.5 M guanidine thiocyanate (GuSCN) in TE buffer, pH 8;

4% polyethylene glycol 8000;

0.24 M NaCl;

0.14 M NaOAC;

0.2% of dithioerythritol (DTE);

0.4% Na₂SO₃;

17.6 μg/ml polyadenylic acid 5′ (PolyA);

0.02% sodium dodecyl sulfate (SDS);

0.4% Tween® 20 detergent (polysorbate 20); and

nuclease free water.

The amount of each reagent listed may vary slightly, such as vary by no more than 5%, no more than 4%, no more than 3%, no more than 2%, no more than 1%, no more than 0.5%, or no more than 0.1%.

In some examples, all or some of the polyethylene glycol 8000 is replaced with a sugar, such as sucrose, glucose, fructose, maltose, or dextrose, for example at a concentration of 0.5% to 5%, such as 1-4%, 2-4%, 3-4%, such as 1%, 2%, 3%, or 4% sugar.

In some examples, the lysis buffer further includes proteinase K, for example at a final concentration of 0.5% to 15%, such as 1% to 12%, 5% to 10%, such as 10%: In some examples, proteinase K is included in the buffer if parasitic nucleic acids (e.g., nucleic acids from Cryptosporidium or Giardia cysts or oocysts) are to be stored in the buffer or on the solid support containing dried lysis buffer.

### Solid Supports and Kits

The present disclosure provides solid supports containing dried disclosed lysis buffer that can be used to store nucleic acid molecules, for example from one or more pathogens. In some examples, the solid supports allow both DNA and RNA, for example from different pathogens, to be stored and extracted. For example, the solid support can allow storage of nucleic acid molecules from a DNA virus and an RNA virus on a single support (e.g., can store DNA and RNA on the same support). In one example, the solid support can allow storage of nucleic acid molecules from multiple pathogens, such as both viruses and bacteria, two or more different viruses (such as at least 2, at least 3, at least 4 or at least 5 different viruses), two or more different bacteria (such as at least 2, at least 3, at least 4 or at least 5 different bacteria), two or more different parasites (such as at least 2, at least 3, at least 4 or at least 5 different parasites), or combinations thereof, on a single support. In one example, the solid support can allow storage of nucleic acid molecules from multiple pathogens present in a stool sample, such as a multiple enteropathogens, for example norovirus, rotavirus, enterovirus, and/or parasites. In one example, the solid support can allow storage of nucleic acid molecules from multiple pathogens present in a respiratory swab sample for example influenza, rhinovirus, RSV, and/or adenovirus.

In some examples, the lysis buffer disclosed herein is applied or contacted with the solid support, and allowed to dry, for example at ambient temperature (such as 18° C. to 25° C., 20° C. to 25° C., 23° C. to 26° C., for example, 22° C., 23° C., 24° C., 25° C. or 26° C.), for example for at least 2 hours, at least 3 hours, at least 4 hours, or at least 6 hours. The solid support can also be also dried more quickly (e.g., 5 to 10 minutes) at a higher temperature, for example 50° C. to 60° C. or 54 to 58° C., such as 56° C., or using a hair dryer. In one example, 60 μl of lysis buffer is applied evenly to a 16 mm circular disk/card.

The solid support containing dried lysis buffer can further include one or more control nucleic acid molecules, such as one or more positive control nucleic acid molecules, one or more negative control nucleic acid molecules, or combinations thereof. Ideally, control nucleic acid molecules do not interact with target pathogen nucleic acid molecules. In one example, the solid support includes a positive control nucleic acid molecule. Such positive controls can be used to allow a user to confirm that nucleic acid molecules were efficiently extracted from the solid support, to confirm that the analysis of the nucleic acid molecules performed properly (e.g., to confirm that the PCR or RT-PCR was not inhibited), or combinations thereof. In some examples, the positive control nucleic acid molecule includes one from bacteriophage MS2 for RNA and PhiX 174 phage for DNA. In some examples, the positive control nucleic acid molecule includes a synthetic nucleic acid molecule, such as one of at least 50 nt, at least 100 nt, or at least 200 nt, such as 100-500 nt, 100-250 nt, such 200 nt. Such control nucleic acid molecules can be applied to the solid support with the buffer, and allowed to dry on the solid support in the same manner. In other examples, the control nucleic acid molecules can be applied to the solid support with the test samples to be analyzed.

The solid support containing dried lysis buffer can further include one or more bacterial, viral, and/or parasitic nucleic acid molecules obtained from a sample. For example, a sample that includes one or more bacteria, viruses, and/or parasites can be applied to the solid support, under conditions that allow the nucleic acid molecules in the sample adhered to the solid support. In some examples, the sample is applied to the solid support directly. In other examples, the sample is first concentrated or diluted (e.g., a stool sample diluted to 10% or less) prior to application to the solid support.

The solid support can be composed of any suitable material, such as cellulose, nitrocellulose, nylon, cotton, silk, polyvinylpyrrolidone (PVPP), glass fiber, cardboard, or plastic. The solid support can be any desired shape, such as circular (such as a disk), oval, square, or rectangular. In one example, the solid support comprises nitrocellulose. In one example, the solid support comprises cellulose, such as cellulose based Whatman-grade 17chr filter paper, such as 16 mm disks thereof.

Also provided are kits that include one or more solid supports disclosed herein, such as at least 5, at least 10, at least 50 or at least 100 of such solid supports. In some examples the solid supports (individually or in multiples) are present in a container, such as a sealable plastic bag. The kits can include other materials, such as one or more of a desiccant, syringe(s), envelope(s), pipette(s), needle(s), plastic bag(s) (e.g., for individual specimen storage/shipping), swabs, gloves, forceps, absorbent pad, paper wipes, sample vials or containers, and one or more positive controls (e.g., MS2 nucleic acid for RNA and PhiX 174 phage nucleic acid for DNA, which can be in a vial or container).

### Storage, Transport, and Analysis of Pathogen Nucleic Acid Molecules

The disclosed solid supports containing the disclosed lysis buffer can be used to stabilize and store nucleic acid molecules from one or more pathogens for future analysis. In some examples, such methods also inactivate pathogens present in the sample. Nucleic acid molecules from one or more pathogens can be stored on the solid support at ambient temperature for at least 5 days, at least 7 days, or at least 14 days, at least 1 month, or at least 3 months (such as 7 days, 14 days, or 30 days), prior to analysis of the nucleic acid molecules from one or more pathogens. During this period, the nucleic acid molecules from one or more pathogens can be transported, for example to a laboratory for analysis, without the need for refrigeration or other cooling (e.g., the solid support containing nucleic acid molecules from one or more pathogens in some examples is not exposed to temperatures at or below 4° C., such as at or below −20° C.).

In other examples, samples are combined directly with the lysis buffer and stored. In this example, a solid support is not used. Instead, the combination of the sample and lysis buffer allows for inactivation of pathogens in the example, and storage of nucleic acid molecules in the sample. Nucleic acid molecules from one or more pathogens stored in the liquid lysis buffer can be stored at ambient temperature for at least 5 days, at least 7 days, or at least 14 days, at least 1 month, or at least 3 months (such as 7 days, 14 days, or 30 days), prior to analysis of the nucleic acid molecules from one or more pathogens. During this period, the nucleic acid molecules from one or more pathogens can be transported, for example to a laboratory for analysis, without the need for refrigeration or other cooling (e.g., the liquid containing nucleic acid molecules from one or more pathogens in some examples is not exposed to temperatures at or below 4° C., such as at or below −20° C.). The nucleic acids in the liquid lysis buffer can be analyzed, for example by sequencing or PCR.

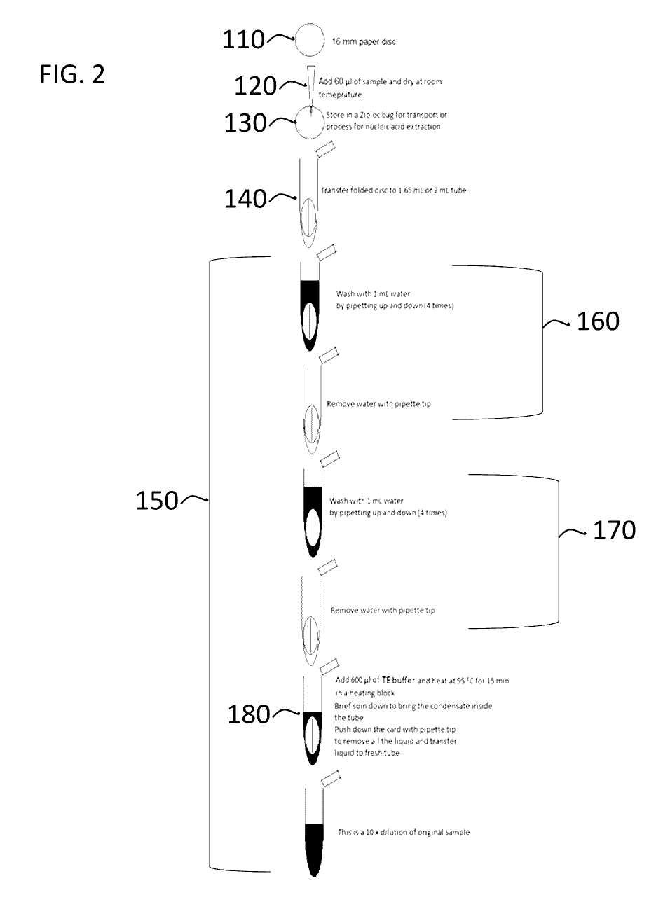

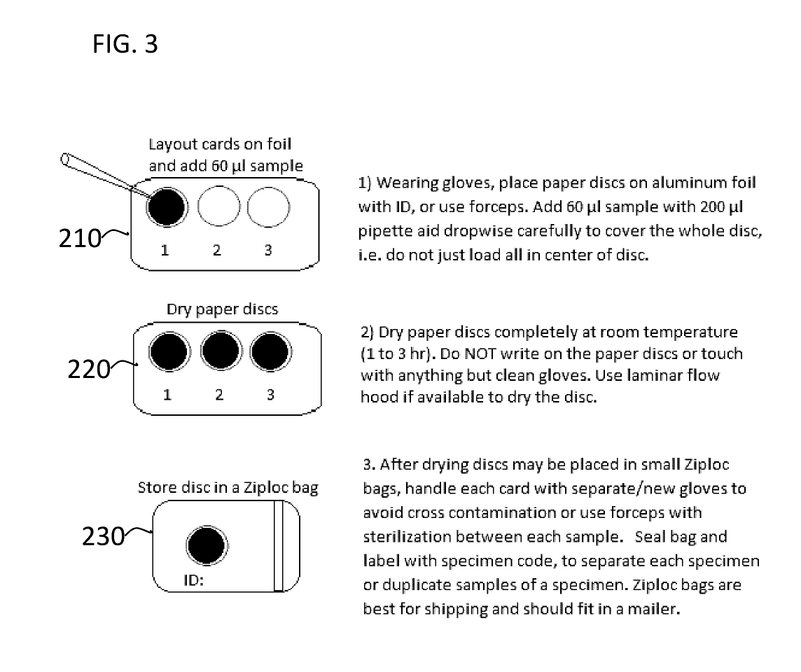

Provided herein are methods of analyzing nucleic acid molecules from the one or more pathogens. A summary of the method is provided in FIG. 2. As shown in FIGS. 2 and 3, such methods can include contacting a solid support containing dried disclosed lysis buffer 110 with a sample 120, wherein the sample contains or is suspected of containing one or more pathogens. For example, for a 16 mm solid support (e.g., nitrocellulose), 60 μl of sample (e.g., a 10% stool suspension) is applied to the solid support using a 200 μl pipette (see FIG. 3, 210). The sample is added dropwise to cover the whole solid support (e.g., it is not all applied to the center of the solid support). In some examples, the sample is allowed to air dry (for example at ambient temperature, preferably in a non-humid area) 130 on the solid support. During drying, the solid support can be placed on aluminum foil or other surface with labels to identify each solid support containing a sample (see FIG. 3, 220). After drying, each solid support can be placed in an individual sealable plastic bag or microfuge tube (e.g., wherein the bag or tube is labeled to permit identification of the sample), 140 (see FIG. 3, 230). Optionally, a desiccant can be included in the bag or tube. Each sample-containing solid support can be handled carefully to avoid cross contamination.

Once applied, the nucleic acid molecules from the one or more pathogens can be stored on the solid support for a period of time, such as at least 3 days, at least 5 days, at least 7 days, at least 14 days, at least 30 days, at least 60 days, at least 90 days, or at least 120 days. In some examples, the nucleic acid molecules from the one or more pathogens can be stored on the solid support at ambient temperatures, such as at 18° C.-40° C., such as 20° C.-35° C., 20° C.-30° C. or 20° C.-22° C. In some examples, the nucleic acid molecules from the one or more pathogens are not exposed to refrigeration or freezing, such as temperatures at or below 4° C., such as at or below −20° C., such as 0° C. to 4° C., or −80° C. to 4° C.

The sample-containing solid support can be stored and/or transported to a diagnostic laboratory, for example via mail. Once at the diagnostic laboratory, the nucleic acid molecules on the solid support are extracted from the solid support and analyzed. For example, as shown in FIG. 2, the disclosed methods include extracting the nucleic acid molecules from the solid support 150, wherein the nucleic acid molecules include nucleic acid molecules from the one or more pathogens. Extraction 150 can include contacting or washing the solid support with water (e.g., nuclease-free water) one or more times 160, 170. In one example, the solid support is washed 2, 3, 4, or 5 times with water, and then the water is removed and buffer added 180, such as a Tris-EDTA (TE) buffer (10 mM Tris, pH 8, 1 mM EDTA) or other suitable nucleic acid buffer. After washing the solid support, the solid support is subsequently heated in buffer 180 (e.g., 600 μL of buffer), for example at a temperature of 90° C. to 100° C., such as 90° C. to 98° C., 92° C. to 98° C., or 95° C. In some examples, this is performed in a microfuge tube. In some examples, the solid support is heated for at least 5 minutes, at least 10 minutes, at least 15 minutes or at least 30 minutes, such as 15 minutes. Following heating the sample can be centrifuged (e.g., 15 seconds at 8000-12000×g to remove liquid from top cap of tube). If 600 μL of buffer was added, the nucleic acid-containing sample is in 600 μl, or 10× volume of the original 60 μL sample applied to the solid support. One skilled in the art will appreciate that other concentrations may be achieved, depending on the sample starting volume and ending volume. This can be considered when comparing Ct values for recovery.

The extracted nucleic acid molecules, which include nucleic acid molecules from the one or more pathogens (if the test sample contained pathogens), can be analyzed. For example, such nucleic acid molecules from the one or more pathogens can be incubated with appropriate primers and/or probes, buffers, and amplified. For example, the nucleic acid molecules can be qualitatively or quantitatively analyzed using PCR (such as RT-PCR, real time qRT-PCR, real time qPCR, or qRT-PCR). In some examples, the nucleic acid molecules are analyzed using nucleic acid sequencing, for example to detect a target mutation. In some examples, the nucleic acid molecules are analyzed using an array contacting complementary nucleic acid molecules to permit detection of target nucleic acid molecules.

In some examples, the extracted nucleic acid molecules, which include nucleic acid molecules from the one or more pathogens (if the test sample contained pathogens), are further treated prior to their analysis. For example, the extracted nucleic acid molecules can be diluted, or passed over a column or filter (for example to remove reagents that may adversely affect PCR, such as polyphenols). In one example, the column filtration membrane has an approximate pore size of 10-20 μm. In one example, the column is a Zymo-Spin™ IV-HRC column. In one example, a Centricon® centrifugal filter is used (such as one with a 50 kDa cut-off). In one example, an Amicon Ultra-15 centrifugal filter is used.

Exemplary samples include environmental samples, such as a water, air, or soil sample, as well as those obtained from a subject, such as blood sample, urine sample, stool sample, sputum sample, respiratory sample, or saliva sample. In some examples, the sample is not treated prior to application to the solid support. In some examples, the sample is treated prior to application to the solid support, such as filtered, concentrated, or diluted. In some examples, nucleic acid molecules in the sample are isolated or purified and then applied to the solid support.

The nucleic acid molecules from the one or more pathogens applied to the solid support can include DNA, RNA, or both. In addition, the method can detect multiple different types of pathogens on the same solid support, such as both a DNA virus and an RNA virus, both a virus and a bacterium, both a virus and a parasite, and the like. Thus, the nucleic acid molecules from the one or more pathogens applied to the solid support can include nucleic acid molecules from the one or more bacteria, viruses, fungi, and/or parasites. In one example, the nucleic acid molecules from the one or more pathogens applied to the solid support include Flavivirus nucleic acid molecules. In one example, the nucleic acid molecules from the one or more pathogens applied to the solid support include E. coli nucleic acid molecules.

### Example 1

### Lysis Buffer

This example describes the composition of the lysis buffer used the Examples below.

| Chemical | Quantity | |
|---|---|---|
| GuSCN | 132 | gram |
| TE(pH 8) | 50 | mL |
| 20% PEG | 50 | mL |
| NaCl(5M) | 12 | mL |
| NaOAC(3M) pH 5.5 | 12 | mL |
| DTE | 0.5 | g |
| Na₂SO₃ | 1 | g |
| PolyA(2 mg/mL) | 2.2 | ml |
| SDS (20%) | 250 | μl |
| Tween ® 20 detergent | 1 | mL |

Make up final volume to 250 mL by adding NF water

Guanidine thiocyanate (GuSCN) Roche, Cat#1685929, 500 g

### Tris EDTA (TE)Buffer pH 8 Ambion,

Nuclease Free water Ambion
Polyethylene glycol 8000 (PEG)

### 5M Sodium Chloride (NaCl) Ambion, Cat#9760G, 100 mL

3M Sodium acetate pH 5.5 (NaOAc) Ambion, Cat#9740, 100 mL

### Dithioerythritol (DTE)

Sodium sulfite (Na₂SO₃)
Polyadenylic acid 5′ (Poly A) Sigma, Cat# P-9403, 25 mg
Tween 20® detergent Fisher or Sigma # P-9416

### 20% SDS (Fisher # BP1311)

### Example 2

### Storage and Recovery of Pathogen DNA and RNA in Water and Lysis Buffer

This example describes methods used to demonstrate the long-term nucleic acid stabilizing effects of the disclosed lysis buffer, as a liquid storage buffer, as compared to water.

The composition of the lysis buffer was as follows:

4.5 M guanidine thiocyanate (GuSCN) in Tris EDTA (TE) Buffer, pH 8;

4% polyethylene glycol 8000;

0.24 M NaCl;

0.14 M NaOAC;

0.2% of dithioerythritol (DTE);

0.4% Na₂SO₃;

17.6 μg/ml polyadenylic acid 5′ (PolyA);

0.02% sodium dodecyl sulfate (SDS);

0.4% Tween® 20 detergent(polysorbate 20); and

water, such as nuclease free water.

A 100-L tap water sample was concentrated to 25 mL, dechlorinated, and seeded with a suite of microbes including whole viruses (rotavirus, hepatitis A virus and adenovirus), bacteria (Salmonella serovar Typhimurium), parasite oocysts (Cryptosporidium parvum), and “naked” RNA (from MS2 bacteriophage). These seeded water samples were added to the lysis buffer at a 1:1 ratio and stored at two different temperature conditions (4° C. and 35° C.). Seeded water sample controls (no lysis buffer) were also stored at 4° C. and 35° C. Duplicate samples were analyzed by real-time PCR or RT-qPCR at nine time points (days 0, 1, 2, 5, 8, 12, 16, 36 and 416). Real-time PCR and RT-PCR crossing threshold (Ct) values were used to monitor DNA and RNA stability over time.

Individual TaqMan® assays were performed for detection of Salmonella serovar Typhimurium) using the methods provided in Hill et al., Appl Environ Microbiol 73(13):4218-25, 2007 and for detection of Cryptosporidium parvum using the methods provided in Jothikumar et al., J. Med. Microbiol. 57:1099-1105, 2008.

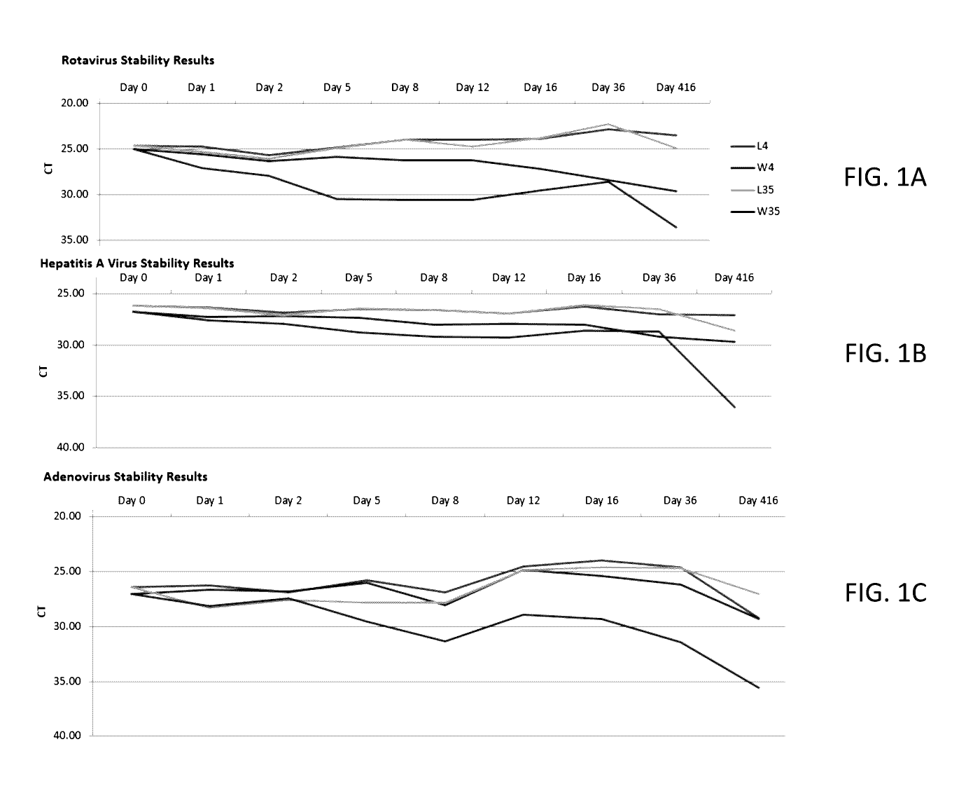

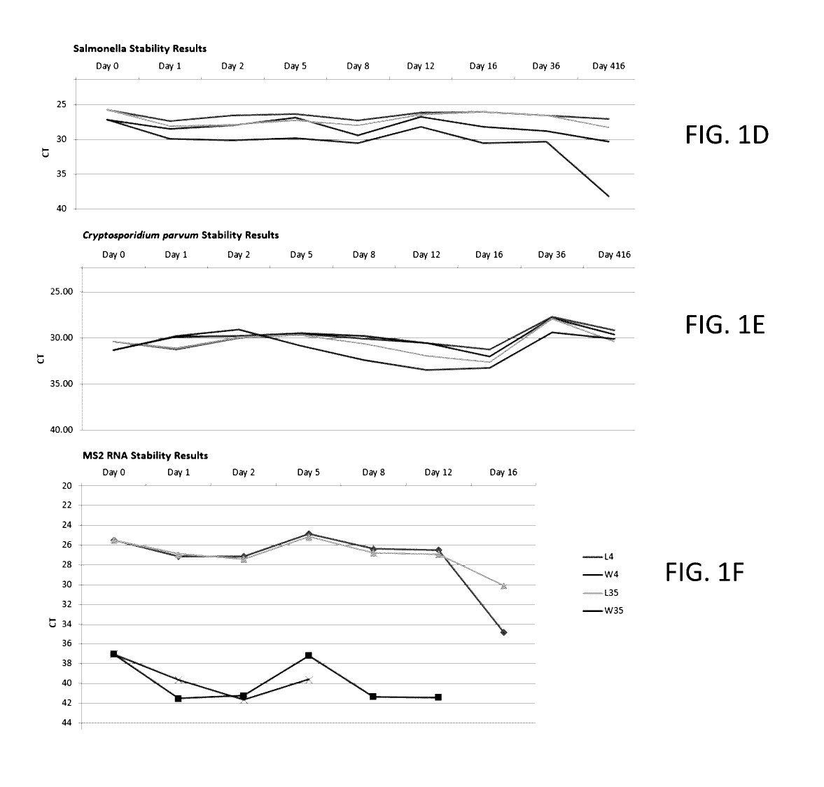

As shown in FIGS. 1A-1E, in a liquid format the lysis buffer provided stability (<2 Ct value increase) of microbial RNA and DNA for ≧416 days when stored at 4° C. and for ≧36 days when stored at 35° C. As shown in FIG. 1F, MS2 naked RNA was stable in the lysis buffer at 4° C. and 35° C. for 12 days, while RNA degraded in control samples (water) within 8 days at 4° C. and 1 day at 35° C. (FIGS. 1A-1E). Thus, the lysis buffer effectively preserved RNA and DNA from a wide variety of microbes in environmental samples stored at 4° C. and 35° C. These results demonstrate that UNEX buffer can serve as an effective storage and transport medium for liquid samples.

### Example 3

### Storage and Recovery of Pathogen DNA and RNA from Solid Support

This example describes methods used to demonstrate the long-term nucleic acid stabilizing effects of the disclosed lysis buffer, as a liquid buffer and in conjunction with a solid matrix. Addition of the lysis buffer to a cellulose card creates a dry, solid matrix for maintaining the stability and integrity of microbial RNA and DNA, which can be efficiently extracted from the card for molecular testing. The lysis buffer contains polyethylene glycol 8000 to facilitate the absorption of nucleic acid molecules on cellulose paper.

The composition of the lysis buffer was as follows:

4.5 M guanidine thiocyanate (GuSCN) in Tris EDTA (TE) Buffer, pH 8;

4% polyethylene glycol 8000;

0.24 M NaCl;

0.14 M NaOAC;

0.2% of dithioerythritol (DTE);

0.4% Na₂SO₃;

17.6 μg/ml polyadenylic acid 5′ (PolyA);

0.02% sodium dodecyl sulfate (SDS);

0.4% polysorbate 20; and

water, such as nuclease free water.

Adenovirus 2 was cultured and plaque assayed in A549 cells to obtain a titer of 1×10⁹ PFU/ml. Hepatitis A virus (HM175-24A) was cultured and plaque assayed FRhK-4 cells to obtain a titer of 1×10⁷ PFU/ml. Cellulose based Whatman-grade 17chr filter paper was soaked in the lysis buffer for 3 hours and air dried. Punches were obtained using a 16 mm hole punch. The 16 mm card was loaded drop wise with 60 μL virus specimen (each card had either HAV or adenovirus). Ten-fold dilutions were loaded (60 μl) onto the individual cards in triplicate. All cards loaded with virus were air dried. The cards were placed directly in a Ziploc® bag without any desiccant and stored at room temperature (˜22° C.).

The nucleic acids were extracted from the cards at the end of drying (time 0), up to 14 days later, as follows. Individual cards were placed in a 1.6-mL microcentrifuge tube, washed twice with water, submerged in 600 μl of water, and transferred to a heating block for at 95° C. for 15 min to release nucleic acids.

The extracted nucleic acids were analyzed using real-time PCR (TaqMan® assays) and Ct values obtained using the methods provided in Jothikumar et al. (Appl Environ Microbiol. 71(6):3131-6, 2005) for Adenovirus, and Jothikumar et al. (Appl Environ Microbiol. 71(6):3359-63, 2005) for hepatitis A.

As shown in Tables 2 and 3, both DNA and RNA from the pathogens were effectively recovered from the cards, up to 14 days after their application to the card. The stability of adenovirus DNA on the buffer paper indicated that viral DNA was stable for 14 days at ambient temperature (Table 2). The stability of hepatitis A virus RNA on the buffer paper indicated that viral RNA was stable for 14 days at ambient temperature (Table 3)

TABLE 2
Recovery and stability of Adenovirus type 2 from the card assessed by real-
time PCR seeded at different dilution levels. Data expressed as Ct values.

| | 10⁴ | 10⁵ | 10⁶ |
|---|---|---|---|
| Dilution | Avg. ± SD | Avg. ± SD | Avg. ± SD |
| Stock | 25.17 ± 0.32 | 28.08 ± 0.66 | 32.03 ± 0.51 |
| 0 h | 29.97 ± 0.51* | 32.30 ± 0.36 | 34.97 ± 0.21 |
| 3 day | 29.90 ± 0.50 | 32.20 ± 0.17 | 35.53 ± 0.29 |
| 7 day | 30.67 ± 0.51 | 33.07 ± 0.31 | 35.67 ± 0.25 |
| 14 day | 31.07 ± 0.61 | 33.23 ± 0.42 | 35.87 ± 0.51 |

*Represents about 50% recovery since it is a 1:10 dilution of stock

TABLE 3
Recovery and stability of hepatitis A virus from the card assessed by
real-time PCR seeded at different dilution levels.

| | 10¹ | 10² | 10³ |
|---|---|---|---|
| Dilution | Avg. ± SD | Avg. ± SD | Avg. ± SD |
| Stock | 23.37 ± 0.47 | 26.77 ± 0.40 | 29.87 ± 0.64 |
| 0 h* | 26.60 ± 0.56* | 29.43 ± 0.59 | 32.83 ± 0.75 |
| 3 day* | 26.27 ± 0.32 | 29.80 ± 0.89 | 32.67 ± 0.45 |
| 7 day* | 26.23 ± 0.51 | 29.63 ± 0.68 | 32.80 ± 0.79 |
| 14 day* | 26.43 ± 0.67 | 29.77 ± 0.81 | 32.53 ± 0.40 |

*A 10 fold dilution of the stock therefore about 100% recovery, assuming 3.3 Ct value per 10X dilution. Essentially all dilutions and time points yielded near 100% recovery of input RNA.

To demonstrate that storage of microbial specimens on the card is safe, the inactivation effectiveness of the card for a diverse set of microbes was examined. Complete inactivation of E. coli (10⁶ CFU/mL), Salmonella enterica serovar Typhimurium (10⁶ CFU/mL), measles virus, and 3 different strains of Middle East Respiratory Syndrome Coronavirus (MERS-CoV) was shown in extracts of the card (disclosed lysis buffer+solid matrix). The long term stability of microbial nucleic acid on the card was also demonstrated (Tables 2 and 3).

The stability and inactivation data demonstrate that the disclosed lysis buffer and card containing such is a safe and effective media for stabilizing nucleic acid (in liquid or solid matrix form) for transport and long-term storage. For example, the buffer and card facilitate ambient temperature nucleic acid transport and long-term storage (e.g., in resource-limited environments).

Thus, the disclosed lysis buffer either in liquid or dried on a card is effective for nucleic acid storage and transport, including at ambient temperatures.

### Example 4

### Comparison of Oocyst Recovery from Buffer and Solid Support

This example provides methods used to compare the recovery of spiked Cryptosporidium parvum oocysts (approximately 60,000 oocysts) from buffer (control) and from a solid support coated with dried lysis buffer,

In one experiment, Cryptosporidium parvum oocysts (approximately 60,000 oocysts) were introduced into 600 μL of TE buffer, and the mixture incubated at 95° C. for 15 minutes in a heating block. 3 μL of the reaction was tested using real-time PCR.

In a parallel experiment, Cryptosporidium parvum oocysts (approximately 60,000 oocysts) were applied to a solid support (16 mm disc) previously incubated with lysis buffer, and the sample dried on the solid support for 3 hours (see Example 3 for disc preparation). The disc was subjected to standard extraction procedure (see Example 3) and the extract generated resuspended in 600 μL of TE buffer, 3 μL of which was tested using real-time PCR.

The real-time PCR reactions were performed as described in Jothikumar et al. (J Med Microbiol 57:1099-1105, 2008).

As shown in Table 4, the amount of Cryptosporidium parvum oocysts recovered was about the same using either method.

TABLE 4
Recovery of spiked Cryptosporidium parvum oocysts in TE buffer
(control) and solid support

| Treatment | C. parvum Ct detected (avg +/− std dev) |
|---|---|
| Oocysts in TE buffer | 34.8 +/− 1.1 |
| Oocysts on disc | 33.1 +/− 0.8 |

### Example 5

### Addition of Positive Control to Test Sample

This example the use of positive controls applied with the test sample to the solid support (card or disc) which includes dried lysis buffer. Such positive controls can be used to determine if PCR inhibitors are present in the test sample.

A solid support (16 mm disc) containing dried lysis buffer was prepared as described in Example 3. A blood sample was spiked with 2.4×10⁷ plaque forming unit (PFU) PhiX 174 DNA (the whole bacteriophage was spiked in; this is a single-stranded, circular, DNA of 5386 nucleotides) and 8.1×10⁷ PFU MS2 RNA (the whole bacteriophage was spiked in; this is a single-stranded, linear, RNA of 3569 nucleotides). 15 μl or 30 μl of the sample was dried on the solid support for 3 hours. The disc was then subjected to the standard extraction procedure (see Example 3) and the nucleic acids recovered resuspended in 600 μL of TE buffer, 3 μL of which was tested using real-time PCR in a total reaction volume of 20 μL of 4×TaqMan® Fast Virus 1-Step MasterMix (Life Technologies, USA). All reactions were performed on Applied Bisosystems 7500 Real-Time PCR System and amplification condition included 5 min at 50° C., 20 sec at 95° C., 45 cycles of 5 sec at 95° C., and 30 sec at 60° C. Fluorescent signals were collected at 60° C. The following primers and probes were used:

MS2 Primers and Probe Sequences

Forward,
(SEQ ID NO: 1)
5′-TGCCATTTTTAATGTCTTTAG-3
Reverse,
(SEQ ID NO: 2)
5′-TGGAATTCCGGCTACCTAC-3′
Probe,
(SEQ ID NO: 3)
5′-/56-FAM/AGACGCTACCATGGCTATCGC/3BHQ_1/-3′

PhiX174 Primers and Probe Sequences

Forward,
(SEQ ID NO: 4)
5′-TCCCAAGAAGCTGTTCAGAATCAGA-3′
Reverse,
(SEQ ID NO: 5)
5′-CACTCCGTGGACAGATTTGTCA-3′
Probe,
(SEQ ID NO: 6)
5′-/56-FAM/TGAGCCGCAACTTCGGGATGA/3BHQ_1/-3′

As shown in Table 5, control viral nucleic acid molecules can be extracted in the presence of blood added to the solid support.

TABLE 5
Parallel recovery of PhiX 174 DNA and MS2 RNA from cards
loaded with constant quantity of phages

| | MS2 Ct | PhiX 174 Ct |
|---|---|---|
| card + 15 μl blood spotted + controls | 22.4 +/− 0.57* | 17.8 +/− 0.14* |
| card + 30 μl blood spotted + controls | 24.73 +/− 0.38^{#} | 18.03 +/− 0.15^{#} |

*Replicate and
^{#}Triplicate

### Example 6

### Comparison of Lysis Buffer Reagents

This example the use of different lysis buffer reagents on the ability to effectively recover nucleic acid molecules on the solid support. The positive controls described in Example 5 were used as the samples (PhiX 174 (DNA bacteriophage, single-stranded, circular, DNA 5386 nucleotides) and MS2 (RNA bacteriophage single-stranded, linear, 3569 nucleotide), to represent DNA and RNA viruses.

A solid support (16 mm disc) containing dried lysis buffer was prepared as described in Example 3. However, different lysis buffer compositions were tested as follows:
Lysis Buffer+1% PEG (less PEG than the buffer shown in Example 1)
Lysis Buffer+2% PEG (less PEG than the buffer shown in Example 1)
Lysis Buffer+4% PEG (this is the buffer shown in Example 1)
Lysis Buffer+1% trehalose (buffer shown in Example 1 without PEG, but with 1% trehalose instead)
Lysis Buffer+2% trehalose (buffer shown in Example 1 without PEG, but with 2% trehalose instead)
Lysis Buffer (buffer shown in Example 1 BUT without PEG)

Stock containing 2.4×10⁷ PFU PhiX 174 DNA and 8.1×10⁷ PFU MS2 RNA was dried on the solid support for 3 hours. The disc was then subjected to the standard extraction procedure (see Example 3) and the nucleic acids recovered resuspended in 600 μL of TE buffer, 3 μL of which was tested using real-time PCR and the primers and probes in Example 5. The results are shown in Table 6. Thus, in some examples, the lysis buffer used in the disclosed methods does not include PEG or a sugar, but allows for preservation and/or transport of nucleic acid molecules on a solid support.

TABLE 6
Comparison of lysis buffers

| | MS2 Ct detected | PhIX174 Ct detected |
|---|---|---|
| Treatment card | (avg +/− std dev) | (avg +/− std dev) |
| UNEX + 1% PEG | 16.7 +/− 0 | 20.4 +/− 0.2 |
| UNEX + 2% PEG | 16.6 +/− 0 | 20.2 +/− 0.4 |
| UNEX + 4% PEG | 16.2 +/− .3 | 20.2 +/− 0.4 |
| UNEX + 1% | 16.7 +/− 0.1 | 19.6 +/− 0.7 |
| Trehalose | | |
| UNEX + 2% PEG + | 16.5 +/− 0.1 | 20.7 +/− 0.6 |
| 0.5% Trehalose | | |
| UNEX only | 17.4 +/− 0.9 | 21.0 +/− 0.6 |

### Example 7

### Addition of Internal Control

This example describes methods that can be used to generate a solid support (card) which includes an internal control, in addition to the lysis buffer. Such a control provides information on inhibitors to PCR that may be present in the test sample.

Solid supports can be prepared as follows. Lysis buffer (Example 1) can be spiked with one or more controls, such as a known amount of the MS2 RNA and/or PhiX 174 DNA described above (e.g., 1 to 10×10⁷ PFU of each). The lysis buffer containing the control nucleic acid molecules is applied to the solid support and allowed to dry as described in example 3.

### Example 8

### Recovery of Control Nucleic Acids and Pathogen Nucleic Acids

This example describes methods used to show parallel recover of norovirus RNA and MS2 RNA from solid supports (cards) containing a 10% stool sample and a controlled amount of MS2.

16 mm discs were coated with lysis buffer as described in Example 3. Subsequently, after the buffer dried, 60 μL of a 10% stool sample containing 29 Ct of MS2 was applied to the discs and allowed to dry. Three different stool samples were analyzed. Duplicate discs were made for each sample. The sample-containing discs were stored from 0 hrs (used immediately after drying), 2 weeks, or 1 month at ambient temperature in a sealable plastic bag. The discs were washed in nuclease free water, and nucleic acids extracted in 600 μL of TE buffer at 95° C. for 15 minutes. The extracted nucleic acids were analyzed by RT-qPCR performed by method of Vega et al. (US. Emerg Infect Dis. 17(8):1389-95, 2011).

As shown in Table 7, both norovirus RNA present in the stool sample and the MS2 control RNA added to the sample were detected, even following 1 month of storage on the disc. These selected samples demonstrated results with about 20 different Norovirus stools loaded onto cards in which a small portion have reduced detection which is corroborated by the reduction in MS2 RNA detection in the presence of the particular stool. Sample 4809 is an example of inhibition. This is likely due to RT-qPCR inhibitors present in the stool sample. Stools are complex and variable clinical samples known to contain inhibitors of PCR. However, the internal standard could also reflect good extraction protocol. If the nucleic acid extraction was not performed correctly, the recovery of internal standard would be reduced, resulting in a higher Ct value as is also seen with inhibition of the molecular reactions. The more common event is inhibition. Using columns (such as the Zymo-Spin™ IV-HRC column), it was observed that inhibition was reduced when the nucleic acid is passed purified on the column. These columns could also be used to concentrate the nucleic acid samples for increased detection. Ct recovery was improved similar to that seen with dilutions of 1:10 of the nucleic or greater. Thus from this data, further treatment or dilution of the nucleic acid extract obtained from the solid support can be performed to reduce the inhibition and enhance detection of the target pathogen nucleic acid molecule.

TABLE 7
Recovery of pathogen and control nucleic acid molecules from
the same solid support.

| STOOL SAMPLE | | M52 CT WITH | M52 |
|---|---|---|---|
| CT LOADED | NOROVIUS | STOOL | (NO STOOL) |
| TIME ON CARD | CT | (29 CT LOADED) | 29 CT LOADED |
| 4809 | 27.0 | 34.0 | 28 |
| (20 CT) | | | |
| 0 TIME | | | |
| 2 WEEKS | 24.0 | 30.0 | 27 |
| 1 MONTH | 25.0 | 32.0 | 28 |
| 1842 | 30.6 | 28.0 | 28 |
| (31 CT) | | | |
| 0 TIME | | | |
| 2 WEEKS | 28.3 | 27.5 | 27 |
| 1 MONTH | 30.6 | 27.2 | 28 |
| 4810 | 27.9 | 29.0 | 28 |
| (26.5 CT) | | | |
| 0 TIME | | | |
| 2 WEEKS | 27.1 | 27.0 | 27 |
| 1 MONTH | 28.9 | 30.9 | 28 |

### Example 9

### Lysis Buffer can Inactivate Infectivity of Viruses

This example provides methods used to demonstrate inactivation of Hepatitis A Virus (HAV) and adenovirus on a card containing dried lysis buffer. It is important that the virus (or other pathogen) on the card is completely inactivated and that infectious virus cannot be obtained from the card. Shipment of the cards containing pathogens (as is recommended for FTA cards) in a regular mail with no warnings requires this information.

To demonstrate that infectious HAV and Adenovirus cannot be recovered from a card containing dried lysis buffer, but infectious virus can be recovered from untreated paper, the following methods were used.

### Hepatitis A Virus (HAV)

60 μl virus (HAV clone 24A titer 1.26E+07 TCID₅₀/ml (cell culture prepared virus), see Cromeans et al., J Gen Virol. 70 (Pt 8):2051-62, 1989) containing 6+E5 TCID₅₀/ml was added to each 16 mm disc previously treated with lysis buffer as described in Example 3, and allowed to dry for 3 hours. The discs were individually placed into separate microfuge tubes, then washed once with water, and 600 μl DMEM 2% FBS (cell culture media) added. The tubes were vortexed, incubated 37° C. for 1 hour with vortexing four times. The supernatant from obtained from the card suspended in DMEM+2% FBS was diluted with DMEM+2% FBS, at the indicated dilutions such as 1:2, 1:4, 1:10, 1:100 and 1:1000 for inoculation of each dilution on cell culture. Each well of a 24 well plate containing FrHK-4 cells was inoculated with 0.1 ml, 5 replicates each.

Simultaneously, HAV was applied to an untreated card (no lysis buffer added) at dilutions of 1:2, 1:4, 10¹, 10² and 10³. The virus was allowed to dry on the card. The control untreated card was treated exactly the same as the lysis buffer treated card, but with no addition of lysis buffer. The resulting eluate from the non-treated card had a TCID₅₀ of 1.21E+05, compared to the 6E+05 inoculated. This demonstrates that infectious virus can be recovered from the card material not treated with lysis buffer.

Results from the lysis buffer treated card were complicated by the fact that, although washed with water, some residual of chemicals remained to be eluted in the DMEM+2% that were toxic to the cell cultures which were inoculated to measure the potential infectious virus. Therefore dilutions of lower than 1:100 could not be evaluated, the cells were destroyed by toxicity before the virus could grow as this is a 7-10 day assay. However, results were obtained from the 1:100 dilutions without toxicity indicating that no infectious virus was present at this concentration. Twelve of twelve inoculated wells (with the 1:100 dilution) were positive for virus with cytopathic effect in the controls, whereas no wells were positive for virus infection (as measured by cytopathic effect), in the eluate from the lysis buffer treated card.

A second approach was used to evaluate whether even one infectious particle could be eluted from the lysis buffer treated card and to show that the positive control of the untreated card has infectious virus that can be eluted. Furthermore inactivation of HAV in the liquid buffer was evaluated. A common method to exchange the solution in which viruses are suspended is use of columns (MW cutoff 50K). Amicon Ultra-15 centrifugal filter units were used to exchange completely the eluate of viruses from the treated and untreated cards in addition to a liquid suspension of viruses and lysis buffer (per FIG. 1 method). The experiments were performed as follows. A liquid solution of 120 μl HAV stock and 120 μl lysis buffer or 120 μl HAV+MEM only were made, each was combined with 10 ml of MEM in an Amicon ultra-15 centrifugal filter units for processing and then reconstituted to 240 μl final volume (same as starting volume) to infect each t25 flask FRHk-4 cells. Solid support experiments were performed as described above with the addition of 60 μl of HAV (undiluted stock) to UNEX cards and to untreated cards. The eluted 600 μl from a card was treated in the ultra-15 centrifugal filter also with the addition of 10 ml MEM and reconstituted to 600 μl for addition of the complete sample to the FRhK-4 cells.

At day 5, the cells incubated in MEM only were normal. The HAV control (the eluate from the untreated card) exhibited-50% cytopathic effect, classic for HAV infection. Other inoculations showed no CPE.

At day 7, the liquid HAV control had a 100% cytopathic effect, indicating virus recovery (e.g., virus was not lost on the Amicon exchange column). Liquid lysis buffer treatment showed no HAV cytopathic effect to indicate presence of any infectious virus. Control card (no lysis buffer) HAV exhibited a 50% cytopathic effect. But on the lysis buffer treated card, HAV exhibited no CPE, indicating virus inactivation.

At day 10, the eluate from the untreated cards that had been processed in the Amicon centrifugal filter unit by the same as that from the lysis buffer treated cards yielded 100% cytopathic effect on the flasks. In stark contrast, no cytopathic effect was seen on the flasks inoculated with the eluate from lysis buffer liquid treated liquid virus, also processed by the same method. This indicates no infectious virus was present in the eluate obtained from the lysis buffer treated card.

### Adenovirus 2

The same method described first above for HAV was used to evaluate Adenovirus 2, that is 60 μl virus stock was added to untreated cards and to lysis buffer treated cards and dried for 3 hours. Cards were eluted with DMEM+2% FBS at 37° C. after a water wash of the card. Dilutions of 1:2, 1:4 and 1:10 were evaluated on A549 cells, commonly used for adenovirus 2 cultivation.

At 2 days post infection, the dilutions of 1:2 and 1:4 of the eluate were toxic to the cell culture and could not be further evaluated. At 6 days post infection, 10⁴ of inoculum was recovered from the untreated card, therefore there was a 10³ loss on the card recovery. At 6 day post infection, eluate from the lysis buffer treated card gave no CPE at up to when diluted 1:10, therefore AdV was inactivated by 10⁷ pfu.

In summary, no infectious HAV or adenovirus was recovered from the cards treated with the disclosed lysis buffer. In contrast, infectious virus was recovered from corresponding non-treated cards and or liquid control samples. This indicates that the cards do not contain infectious particles and can be shipped in the mail without the label of pathogen. The lysis buffer inactivated HAV in liquid and card form completely and adenovirus 2 was inactivated on the card by 1E+07.

### Example 10

### Testing of Additional Samples

Two field visits have been conducted (in Ghana and Ethiopia) and several water sources have been analyzed. Source types include boreholes, surface water, public taps, and unprotected dug wells. Replicate samples from each source were analyzed as follows: Colilert-18® incubated at standard conditions (35° C. for 18-22 hrs); CBT incubated at ambient temperature for 24 hrs; CBT incubated at ambient temperature for 48 hrs; and CBT incubated at 35° C. for 24 hrs. Results obtained from CBT tests were compared with those from Colilert-18®, which is considered a gold standard method for E. coli quantification. Aliquots of enrichment broths from a subset of Colilert-18® and CBT tests (both positive and negative for E. coli) were preserved on the disclosed cards with dried lysis buffer and shipped to the CDC for molecular analyses.

DNA samples were stored on the treated cards, followed by nucleic acid extraction and qPCR. A TaqMan® assay was conducted using 250 nM each forward and reverse primer and 100 nM probe in 25 μL reaction volumes using ABI Environmental Master Mix 2.0 and 6 μL of template extract.

A duplex PCR assay targeting uidA (T6)/tnaA (T10) genes was used to confirm E. coli from the card. Probes were labeled with FAM or Cy5. Real-time PCR cycling conditions consisted of one cycle of denaturation at 95° C. for 10 minutes, followed by 45 cycles of denaturation at 95° C. for 5 seconds, and annealing, extension and fluorescence acquisition at 60° C. for 30 seconds.

T6ECF,
(SEQ ID NO: 7)
CGGGACTTTGCAAGTGGTGAA
T6ECR,
(SEQ ID NO: 8)
ACGCACAGTTCATAGAGATAACCT
T6ECP,
(SEQ ID NO: 9)
FAM-5CCCACCTCTGGCAACCGGGT-3BHQ1
T10ECF,
(SEQ ID NO: 10)
GGACCATCGAGCAGATCACC
T10ECR,
(SEQ ID NO: 11)
CCCATCGGCACCATCGCA
T10ECP,
(SEQ ID NO: 12)
Cy5-5TGCCGATATGCTGGCGATGTCCGCCAA-3BHQ2

This allowed the sensitivity and specificity of ambient temperature incubation to be determined. As shown in Table 8, while the compartment bag test (CBT) indicated a high number of E. coli true positive results, at cooler ambient incubation temperatures (average 21.8° C.), the CBT indicated a high number of false negative results. The specificity refers to the liquid medium (broth) used for the growth of bacteria. Thus, the disclosed solid supports with dried lysis buffer can be used to detect E. coli.

TABLE 8
Sample DNA storage using cards treated with lysis buffer

| | Ghana (avg 30.2° C.) | | Ethiopia (avg 21.8° C.) | |
|---|---|---|---|---|
| | PCR+ | PCR− | PCR+ | PCR− |
| CBT+ | 49 | 3 | 26 | 25 |
| CBT− | 2 | 44 | 2 | 14 |
| | Sensitivity | 96% | Sensitivity | 93% |
| | Specificity | 94% | Specificity | 36% |

Stool samples were analyzed for recovery up to 2 weeks post inoculation of the card.

As shown in Table 9, 50-100% recovery was obtained with all samples. One GI and one GII norovirus were analyzed over three 10-fold dilution series and detection of all samples was obtained up to 1:1000 dilution of the stool preparations. In those cases where recovery was low, in general, detection of the MS2 standard was reduced. MS2 only recovery up to 1 month is excellent and good at 3 months at ambient temperature, showing variation between stool samples. Storage at longer temperatures could be enhanced by cooler storage temperatures and/or desiccant.

TABLE 9
Norovirus 10% stool samples inoculated onto treated card and maintained
at ambient temperature for indicated times

| Clinical samples + | % recovery RNA | % recovery | % recovery |
|---|---|---|---|
| MS2 | 2 wk on card | 1 month | 3 months |
| 4557 | 75 | 65 | 1 |
| 4810 | 100 | 50 | 1 |
| 2299 | 100+ | 100+ | 34 |
| 1842 | 100+ | 100 | 1 |
| 1843 | 68% | 100 | 100 |
| 1844 | 75% | 99 | 85 |
| 8501 | <1% | <1 | 100 |
| MS2 only | 100 | 100 | 52 |

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A lysis buffer, comprising or consisting of:
1 M to 5 M guanidine thiocyanate (GuSCN) in Tris EDTA (TE) Buffer;
0.5% to 4% polyethylene glycol 8000;
0.1 M to 2 M NaCl;
0.05 M to 1 M NaOAC;
0.1% to 1% of dithioerythritol (DTE);
0.1% to 2% Na₂SO₃;
1 μg/ml to 100 μg/ml polyadenylic acid 5′ (PolyA);
0.01% to 0.5% sodium dodecyl sulfate (SDS);
0.1% to 2% polysorbate 20; and
water, such as nuclease free water.

2. The lysis buffer of claim 1, when a volume of 250 ml comprises or consists of:
132 grams of guanidine thiocyanate (GuSCN);
50 mL of Tris-EDTA (TE) Buffer, pH 8;
50 mL of 20% polyethylene glycol 8000;
12 mL of 5M NaCl;
12 mL of 3M NaOAC, pH 5;
0.5 g of dithioerythritol (DTE);
1 g of Na₂SO₃;
2.2 ml of polyadenylic acid 5′ (PolyA, at 2 mg/mL);
250 μl of 20% sodium dodecyl sulfate (SDS);
1 mL of polysorbate 20) and
remaining volume of nuclease free water.

3. The lysis buffer of claim 1, wherein the buffer comprises or consists of:
4.5 M guanidine thiocyanate (GuSCN) in Tris-EDTA (TE) Buffer, pH 8;
4% polyethylene glycol 8000;
0.24 M NaCl;
0.14 M NaOAC;
0.2% of dithioerythritol (DTE);
0.4% Na₂SO₃;
17.6 μg/ml polyadenylic acid 5′ (PolyA);
0.02% sodium dodecyl sulfate (SDS);
0.4% polysorbate 20; and
nuclease free water.

4. The lysis buffer of claim 3, further comprising 10% proteinase K.

5. A solid support, comprising:
the lysis buffer of claim 3, dried on the solid support.

6. The solid support of claim 5, further comprising:
one or more control nucleic acid molecules.

7. The solid support of claim 6, wherein the control nucleic acid molecule comprises a positive control nucleic acid molecule.

8. The solid support of claim 7, wherein the positive control nucleic acid molecule comprises an RNA bacteriophage MS2 nucleic acid molecule and/or a DNA bacteriophage PhiX 174 nucleic acid molecule.

9. The solid support of claim 5, further comprising:
bacterial, viral, and/or parasitic nucleic acid molecules obtained from a sample.

10. The solid support of claim 5, wherein the solid support comprises cellulose, nitrocellulose, cardboard, or plastic.

11. A kit comprising:
one or more of the solid supports of claim 5; and
one or more of a desiccant, a syringe, an envelope, a plastic bag, forceps, gloves, a pipette, and a needle.

12. A method of analyzing nucleic acid molecules from the one or more pathogens, comprising:
contacting the solid support of claim 5 with a sample, wherein the sample comprises or is suspected of comprising one or more pathogens;
extracting the nucleic acid molecules from the solid support, wherein the nucleic acid molecules comprise nucleic acid molecules from the one or more pathogens; and
analyzing the extracted nucleic acid molecules from the one or more pathogens.

13. The method of claim 12, wherein the nucleic acid molecules from the one or more pathogens comprise DNA, RNA, or both.

14. The method of 12, wherein the nucleic acid molecules from the one or more pathogens are bacterial, viral, and/or parasitic nucleic acid molecules.

15. The method of 12, wherein the nucleic acid molecules from the one or more pathogens comprise viral DNA and viral RNA.

16. The method of 14, wherein the nucleic acid molecules from the one or more pathogens comprise Flavivirus nucleic acid molecules.

17. The method of 14, wherein the nucleic acid molecules from the one or more pathogens comprise E. coli nucleic acid molecules.

18. The method of claim 12, wherein the solid support comprises cellulose, nitrocellulose, cardboard, or plastic.

19. The method of claim 12, wherein the sample is a water sample, blood sample, urine sample, stool sample, sputum sample, respiratory sample, or saliva sample.

20. The method of claim 12, wherein extracting the nucleic acid molecules from the solid support comprises heating the solid support in water or buffer at a temperature of 90° C. to 100° C.
